(19) United States
(12) Patent Application Publication
(10) Pub. No.: US 2017/0027507 A1
(43) Pub. Date: Feb. 2, 2017

(54) METHOD AND APPARATUS FOR MEASURING PHYSICAL CONDITION BY USING HEART RATE RECOVERY RATE

(71) Applicant: JoiiUp Technology Inc., Zhubei City (TW)

(72) Inventors: Geng-Chang Yeh, Zhubei City (TW); Pei-Lin Tsai, Zhubei City (TW); Ming Chao, Zhubei City (TW); Kenn Slagter, Zhubei City (TW); Fueng-Ho Chen, Zhubei City (TW)

(73) Assignee: JoiiUp Technology Inc., Zhubei City (TW)

(21) Appl. No.: 14/843,428

(22) Filed: Sep. 2, 2015

(30) Foreign Application Priority Data
Jul. 30, 2015 (TW) 104124748

(51) Int. Cl. A61B 5/00; A61B 5/024

(52) U.S. Cl. CPC A61B 5/4884; A61B 5/024; A61B 5/7225; A61B 5/7282; A61B 5/40

(57) ABSTRACT

A method for measuring a physical condition of an individual by using a post-exercise heart rate recovery rate is provided and, has steps of: acquiring a heart rate of the individual during a recovery period after a physical activity of the individual; calculating a heart rates recovery rate of the individual using a nonlinear regression equation, which includes a parameter representing the rate of heart rate recovery; and determining the physical condition of the individual based upon the parameter. Hereby, the accuracy of measuring the heart rate recovery rate is better than other conventional methods.

### CROSS REFERENCE TO RELATED APPLICATIONS

This application claims priority to Taiwan Application Serial Number 104124748, filed on Jul. 30, 2015, which are herein incorporated by reference.

### FIELD OF THE INVENTION

The present invention relates to a method and an apparatus for measuring a physical condition, and in particular to a method and an apparatus for measuring a physical condition (e.g. the cardiopulmonary function/autonomic nervous regulation condition) by using a heart rate recovery.

### BACKGROUND OF THE INVENTION

The Harvard step test was developed by Professor Brouha et al. (1943) in the Harvard Fatigue Laboratories in the United States, which is a simple and effective method determining the cardiopulmonary function based upon the heart rate recovery rate after step aerobics. In the original method, the subject repeatedly steps up and down with a certain frequency within a specified period of time. The height of each step is 20 inches (50.8 cm), and the stepping frequency is stepping up and down 30 times per minute, and the exercise time is usually 3 minutes, or 5 minutes at most. Three heartbeat numbers are recorded from 1 min to 1 mm 30 sec, from 2 mm to 2 mm 30 sec, and from 3 mm to 3 min 30 sec after the step aerobics during the recovery period. The three heartbeat numbers during the recovery period are used to calculate a physical index. The calculation method of the cardiopulmonary endurance index uses the period of time (sec) of the exercise multiplied by 100 as the numerator, and uses the sum of the three heartbeat numbers multiplied by 2 as the denominator, for determining the human cardiopulmonary endurance and the ability of adjustment and recovery under physical fatigue. The higher the index is, the better the muscular endurance is, and the stronger the cardiopulmonary function is.

It is verified in a great amount of research that the heart rate recovery after exercise is a significant representation of the heart rate regulated by the autonomic nervous system and the cardiopulmonary function. The heart rate recovery rate is one of the important indexes for evaluating the physical conditions, such as the autonomic nervous system regulation and the cardiopulmonary function, and moreover has a positive correlation with the decline of the cardiovascular mortality. During the recovery period, the heart rate declines along a nonlinear curve, but the heart rate recovery rate after exercise is calculated by using a linear regression equation in most conventional techniques, as shown in FIG. 1. Furthermore, the heart rate recovery rate after exercise is calculated by non-continuously measuring the heart rate 1-5 minutes after exercise, so that the measurement accuracy is poor. Therefore, it is necessary to develop a method or an apparatus for continuously measuring the heart rate for a sustained time period after exercise (such as 10 to 15 minutes) and calculating the heart rate recovery rate by using a nonlinear regression equation, thereby accurately evaluating the physical condition (e.g. the cardiopulmonary function/autonomic nervous regulation condition).

### SUMMARY OF THE INVENTION

A primary object of the present invention is to provide a method and an apparatus for measuring a physical condition (e.g. the cardiopulmonary function/autonomic nervous regulation condition), which calculates a heart rate recovery rate after a physical activity by using a nonlinear regression equation for resolving the inaccuracy problem resulting from the calculation of the heart rate recovery rate after physical activity by using a linear regression equation in the prior art.

To achieve the above object and improve the defect of the prior art, the present invention provides a method for measuring a physical condition by using a heart rate recovery rate, comprising steps of:
acquiring a heart rate of an individual during a recovery period after a physical activity conducted by the individual;
calculating a nonlinear regression equation of the heart rate of the individual, which includes a parameter representing the heart rate recovery rate of the individual, based upon the heart rate of the individual; and
determining the physical condition of the individual based upon the parameter.

In an embodiment of the present invention, the nonlinear regression equation comprises an exponential regression equation or a logarithmic equation.

In an embodiment of the present invention, the exponential regression equation is defined as the following equation:

HR(t)=HR_{d}×C^{(-K×t)}+HRᵣ; or

HR=(HR₀−HRᵣ)×C^{(-K×t)}+HRᵣ,
wherein t is time, HR(t) is the heart rate at the time t, HRᵣ is a resting heart rate, HR₀ is a heart rate at the initiation of the recovery period, HR_{d} is a difference between the heart rate at the initiation of the recovery period and the resting heart rate, K is a parameter representing the heart rate recovery rate of the individual, and C is a constant.

In an embodiment of the present invention, a relationship between a heart rate recovery half life and the parameter K representing the heart rate recovery rate of the individual is defined as the following equation:

$${t}_{1 / 2} = \frac{{log}_{c} 2}{K} ,$$

wherein t_{1/2} is the heart rate recovery half life.

In an embodiment of the present invention, wherein the logarithmic regression equation is defined as the following equation:

HR(t)=a×log_{c}t+R,
wherein t is time, HR(t) is the heart rate at the time t, R is a parameter associated with a resting heart rate, a is a parameter representing the heart rate recovery rate of the individual, and C is a constant.

In an embodiment of the present invention, the constant C is a natural logarithm e.

In an embodiment of the present invention, the physical activity conducted by the individual is a step test.

In an embodiment of the present invention, the step of acquiring a heart rate of the individual during a recovery period after a physical activity conducted by the individual comprises:

continuously acquiring the heart rate of the individual during the recovery period after the physical activity conducted by the individual.

In an embodiment of the present invention, the physical condition is the cardiopulmonary function condition or an autonomic nervous control condition.

To achieve the above object and resolve the defect of the prior art, the present invention provides an apparatus for measuring a physical condition by using a heart rate recovery rate, comprising:
a heart rate acquiring unit configured to acquire a heart rate of an individual during a recovery period after a physical activity conducted by the individual, and to output a digitalized signal of the heart rate; and
a heart rate processing unit connected with the heart rate acquiring unit to receive the digitalized signal of the heart rate, to calculate a nonlinear regression equation of the heart rate of the individual, which includes a parameter representing the heart rate recovery rate, based upon the heart rate of the individual, and to determine the physical condition of the individual based upon the parameter.

In an embodiment of the present invention, the nonlinear regression equation comprises an exponential regression equation or a logarithmic equation.

In an embodiment of the present invention, the exponential regression equation is defined as the following equation:

HR(t)=HR_{d}×C^{(-K×t)}+HRᵣ; or

HR=(HR₀−HRᵣ)×C^{(-K×t)}+HRᵣ,
wherein t is time, HR(t) is the heart rate at the time t, HRᵣ is a resting heart rate, HR₀ is a heart rate at the initiation of the recovery period, HR_{d} is a difference between the heart rate at the initiation of the recovery period and the resting heart rate, K is a parameter representing the heart rate recovery rate of the individual, and C is a constant.

In an embodiment of the present invention, a relationship between a heart rate recovery half life and the parameter K representing the heart rate recovery rate of the individual is defined as the following equation:

$${t}_{1 / 2} = \frac{{log}_{c} 2}{K} ,$$

wherein t_{1/2} is the heart rate recovery half life.

In an embodiment of the present invention, the logarithmic regression equation is defined as the following equation:

HR(t)=a×log_{c}t+R,
wherein t is time, HR(t) is the heart rate at the time t, R is a parameter associated with a resting heart rate, a is a parameter representing the heart rate recovery rate of the individual, and C is a constant.

In an embodiment of the present invention, the constant C is a natural logarithm e.

In an embodiment of the present invention, the physical activity conducted by the individual is a step test.

In an embodiment of the present invention, the heart rate acquiring unit continuously acquires the heart rate of the individual during the recovery period after physical activity conducted by the individual.

In an embodiment of the present invention, the apparatus is a wearable apparatus.

In an embodiment of the present invention, the physical condition is the cardiopulmonary function condition or an autonomic nervous control condition.

### BRIEF DESCRIPTION OF THE DRAWINGS

The foregoing aspects and many of the attendant advantages of this invention will become more readily appreciated as the same becomes better understood by reference to the following detailed description, when taken in conjunction with the accompanying drawings, wherein:

FIG. 1 is a schematic graph showing the calculation of a heart rate recovery rate after a physical activity by using a linear regression equation in conventional techniques;

FIG. 2 is a flowchart of steps of a method for measuring a physical condition of an individual by using a heart rate recovery rate after a physical activity in accordance with an embodiment of the invention;

FIG. 3 is a schematic graph showing the calculation of a heart rate recovery rate after a physical activity by using an exponential regression equation in accordance with an embodiment of the invention;

FIG. 4 is a schematic graph showing the calculation of a heart rate recovery rate after a physical activity by using a logarithmic regression equation in accordance with an embodiment of the invention;

FIG. 5A-FIG. 5C are schematic graphs showing the exponential regression equations of three different individuals calculated by using a method of the invention, after the three individuals conduct physical activities with equal intensity for equal periods, and the heart rates thereof are measured;

FIG. 6 is another schematic graph showing the calculation of a heart rate recovery rate after a physical activity by using an exponential regression equation in accordance with an embodiment of the invention; and

FIG. 7 is a structural schematic diagram of an apparatus for measuring a physical condition of an individual by using a heart rate recovery rate after a physical activity in accordance with an embodiment of the invention.

### DETAILED DESCRIPTION OF THE PREFERRED EMBODIMENTS

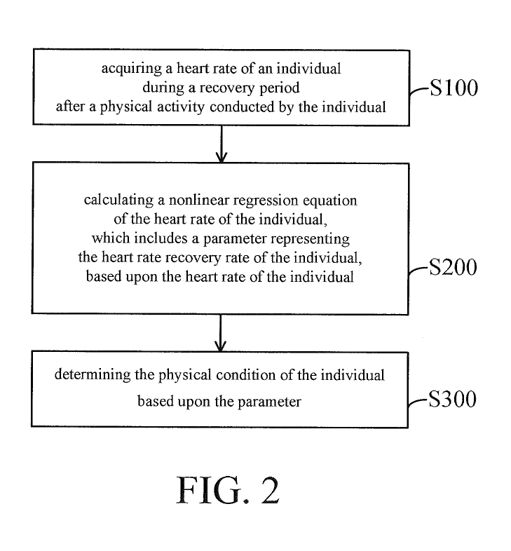

Please refer to FIG. 2, which is a flowchart of steps of a method for measuring a physical condition (e.g. the cardiopulmonary function/autonomic nervous regulation condition) of an individual by using heart rate recovery rate after a physical activity in accordance with an embodiment of the invention. The method for measuring the physical condition of the individual by using the heart rate recovery rate after the physical activity includes the following steps: in step S100, the heart rate of the individual during a recovery period is acquired after the physical activity conducted by the individual; in step S200, a nonlinear regression equation of the heart rate of the individual is calculated, which includes a parameter representing the heart rate recovery rate of the individual, based upon the heart rate of the individual; in step S300, the physical condition of the individual is determined based upon the parameter (e.g. the cardiopulmonary function/autonomic nervous regulation condition).

In the embodiments of the present invention, the physical activity conducted by the individual includes any physical activity which can raise the heart rate, such as step aerobics, hiking, jogging, walking, ball games, dancing, swimming, yoga, boating, etc. The above physical activities are only the exemplary embodiments of the present invention, and should not be regarded as limiting.

In the embodiments of the invention, the method for measuring the heart rates of the individual during the recovery period includes detecting ECG signals, heart sounds and pulses. ECG signals are typically detected by using at least one pair of electrodes, and the ECG signal detection methods can be divided into invasive methods and non-invasive methods. When the method of the present invention is applied to an implantable medical device, the invasive ECG signal measuring methods can be used. When the method of the present invention is applied to a wearable device, the non-invasive ECG signal measuring methods can be used. Due to the convenience of wearable devices, the ECG signals are measured by using the non-invasive ECG signal measuring methods on a wearable device in all the embodiments of the present specification, which does not means that the other methods for measuring the heart rate are not feasible. The above methods for measuring the heart rate are only the exemplary embodiments of the present invention, and should not be regarded as limiting.

The recovery period described in the present invention refers to a period during which the heart rate of an individual after a physical activity begins to decline to a resting heart rate. The recovery period begins at the end of the physical activity, and ends when the heart rate declines to the resting heart rate. The resting heart rate refers to the heart rate of an individual who does not conduct any physical activity, and at this time the least amount of blood per unit of time is pumped by the heart. The resting heart rate of humans is generally ranged from 40 to 100 times per minute, and depends on the individual differences and exercise habits. Generally, individuals having exercise habits have lower heart rates than individuals without exercise habits. This means that the heart thereof can pump more blood than others. The heart rate recovery rate after the physical activity refers to the declining rate of the heart rate of an individual during the recovery period.

The heart rate of an individual can be measured during a period of time in an early phase, a middle phase, or a late phase of the recovery period or during the whole recovery period. The period of time can be 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 minutes, or all numerical ranges included within 1-30 minutes. 10-15 minutes or 3-5 minutes are preferably selected in the embodiments of the present invention. The measuring periods of time above are only the exemplary embodiments of the present invention, and should not be regarded as limiting.

In the present invention, “continuously measuring the heart rate” refers to measuring the heart rate during the period of time multiple times, and the interval of time between each measurement is not more than 10 seconds. In all the embodiments of the present invention, the heart rates are measured one time per second. This is different from the non-continuous measurement in conventional techniques, in which the heart rate is measured one time per minute or every dozen seconds. In conventional techniques, due to few measurements, the accuracy is poor, and only the linear regression equation can be used to calculate the heart rate recovery rate after the physical activity, causing poorer accuracy.

During the recovery period, the heart rate declines along a nonlinear equation, but the heart rate recovery rate after the physical activity is calculated by using a linear regression equation in most conventional techniques, so as to cause poor accuracy. In contrast, in the method or the apparatus of the present invention, the heart rate recovery rate after the physical activity is calculated by using a nonlinear regression, so as to evaluate the physical condition more accurately (e.g. the cardiopulmonary function/autonomic nervous regulation condition). The nonlinear regression equation in the present invention refers to a nonlinear regression equation of a heart rate is calculated based upon the heart rate data measured from an individual. The nonlinear regression equation includes an exponential equation, a logarithmic equation, a trigonometric equations, an elliptic equations, a circular equation, a quadratic equations, a cubic equation, a higher-degree equation and so on. Therefore, all kinds of nonlinear equations can be applied to calculate the heart rate recovery rate and measure the physical activity based upon the disclosure of the present invention. In particular, exponential equations and logarithmic equations conform the decline curve of the heart rate more than other equations, wherein the method for measuring the physical condition by using an exponential regression equation is the best mode. Therefore, exponential regression equations and logarithmic regression equations are used as the exemplary embodiments of the present equation, and should not be regarded as limiting.

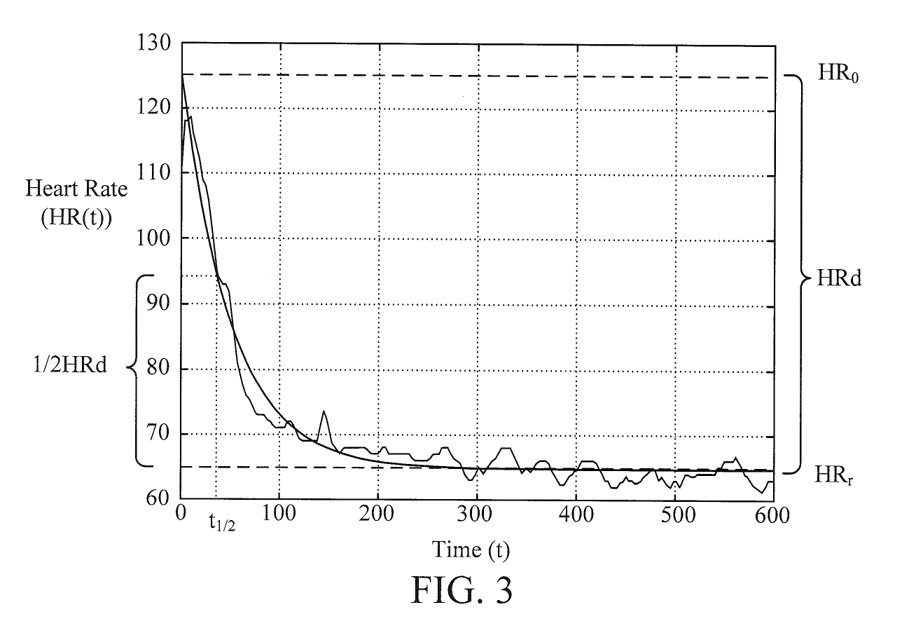

Please refer to FIG. 3, which is a schematic graph showing the calculation of a heart rate recovery rate after a physical activity by using an exponential regression equation in accordance with an embodiment of the invention. The horizontal axis represents time, the vertical axis represents the heart rate, the tortuous curve represents the heart rate data of an individual, and the smooth curve represents the exponential regression equation calculated based on the heart rate data. The exponential regression equation formula is defined as follows:

HR(t)=HR_{d}×C^{(-K×t)}+HRᵣ; or Formula 1:

HR=(HR₀−HRᵣ)×C^{(-K×t)}+HRᵣ Formula 2:

wherein t is time, HR(t) is the heart rate at the time t, HRᵣ is a resting heart rate, HR₀ is a heart rate at the initiation of the recovery period, HR_{d} is a difference between the heart rate at the initiation of the recovery period and the resting heart rate, K is a parameter representing the heart rate recovery rate of the individual, and C is a constant. In this embodiment, the constant C is a natural logarithm e.

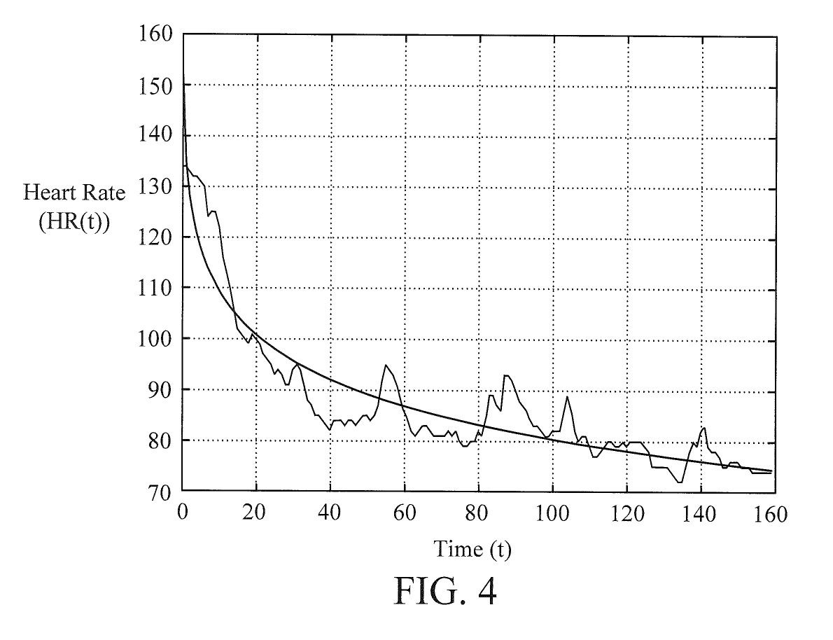

Please refer to FIG. 4, which is a schematic graph showing the calculation of a heart rate recovery rate after a physical activity by using an logarithmic regression equation in accordance with an embodiment of the invention. The horizontal axis represents time, the vertical axis represents the heart rates, the tortuous curve represents the heart rate data of an individual, and the smooth curve represents the logarithmic regression equation calculated based on the heart rate data. The logarithmic regression equation formula is defined as follows:

HR(t)=a×log_{c}t+R Formula 3:

wherein t is time, HR(t) is the heart rate at the time t, R is a parameter associated with a resting heart rate, a is a parameter associated with the heart rate recovery rate of the individual, and C is a constant. In this embodiment, the constant C is a natural logarithm e.

The heart rate recovery half life t_{1/2} after the physical activity refers to the period of time which is required for the heart rate to decline by a half of HR_{d} from HR₀. The relationship between the heart rate recovery half life t_{1/2} after the physical activity and the parameter K representing the heart rate recovery rate of the individual is defined as the following equation:

$${t}_{1 / 2} = \frac{{log}_{c} 2}{K} \qquad Formula 4$$

In the present embodiment, since the constant C is a natural logarithm e, the relationship between the heart rate recovery half life t_{1/2} after the physical activity and the parameter K representing the heart rate recovery rate of the individual is defined as the following equation:

$${t}_{1 / 2} = \frac{ln 2}{K} \qquad Formula 5$$

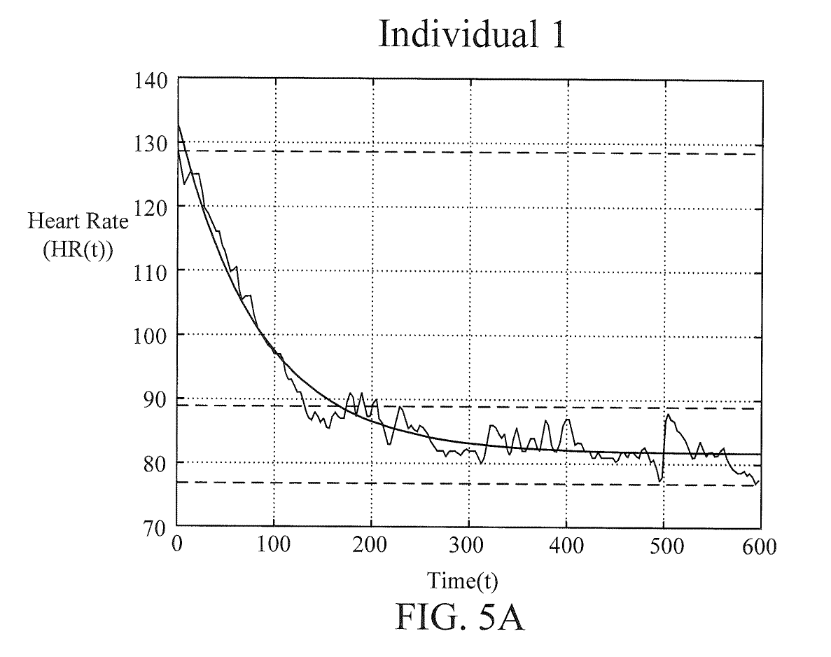

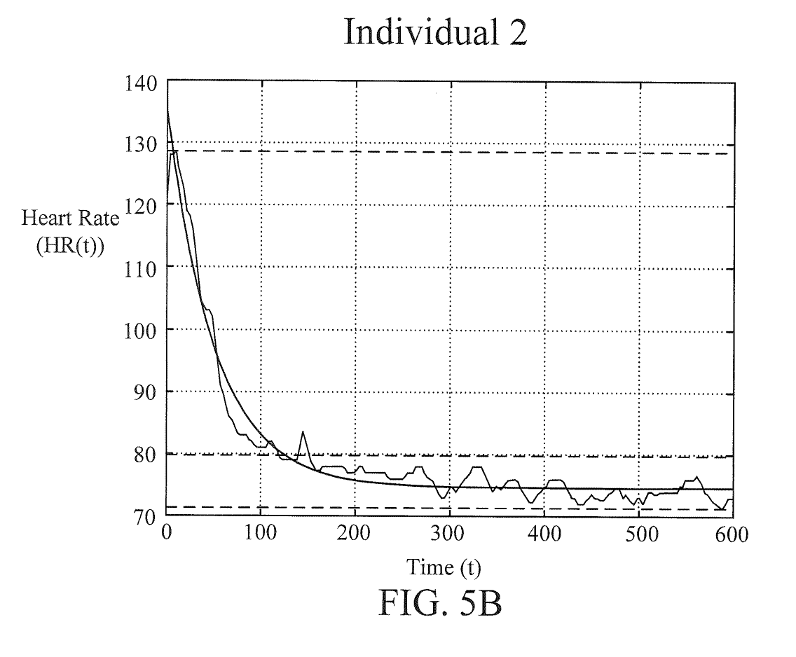

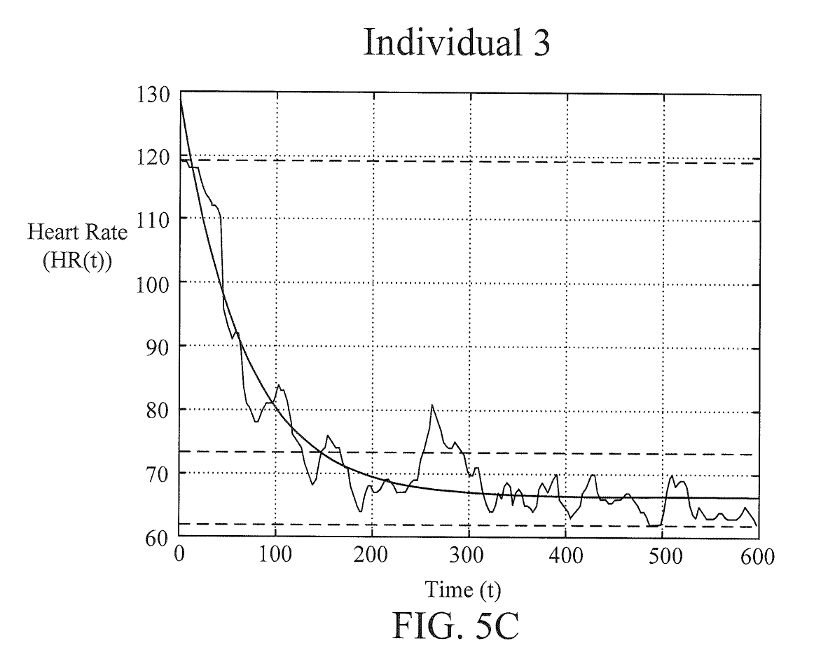

Since K is the parameter representing the heart rate recovery rate of the individual after the physical activity, the greater the value of K is, the higher the heart rate recovery rate is, so that the value of K can be used to determine the physical condition of the individual (e.g. the cardiopulmonary function/autonomic nervous regulation condition). Please refer to FIG. 5A-FIG. 5C, which are schematic graphs showing the exponential regression equations of three different individuals calculated by using a method of the invention, after the three individuals conduct physical activities with equal intensity for equal periods, and their heart rates are measured. The horizontal axis represents time, the vertical axis represents the heart rates, the tortuous curves represent the heart rate data of the individuals, and the smooth curves represent the exponential regression equations calculated based on the heart rate data. Pleas refer to Table 1, which shows the measurement results of the cardiopulmonary function of the three different individuals conducting the physical activities with equal intensity for the equal periods. All of the heart rate at the initiation of the recovery period (HR₀), the parameter (K) representing the heart rate recovery rate of the individual after the physical activity, the heart rate recovery half life (t_{1/2}) after the physical activity, and the R-square, which is a coefficient of determination representing the accuracy, are recorded. In Table 1, the greatest parameter K of the individual 2 represents that the heart rate recovery rate thereof after the physical activity is the highest, and the physical condition thereof (e.g. the cardiopulmonary function/autonomic nervous regulation condition) is the strongest. Hence, in FIG. 5B, the curve of the exponential regression equation of the heart rate has the steepest decline, and the heart rate recovery rate (t_{1/2}) after the physical activity is the shortest. In Table 1, the least parameter K of the individual 3 represents that the heart rate recovery rate thereof after the physical activity is the lowest, and the physical condition thereof (e.g. the cardiopulmonary function/autonomic nervous regulation condition) is the worst. Hence, in FIG. 5A, the curve of the exponential regression equation of the heart rate has the flattest decline, and the heart rate recovery rate (t_{1/2}) after the physical activity is the longest.

TABLE 1

| | | | | R square |
|---|---|---|---|---|
| | HR₀ | K | T_{1/2} | (coefficient of |
| | (time/mm) | (sec⁻¹) | (sec) | determination) |
| Individual 1 | 133.21111 | 0.01181 | 58.69421 | 96.15% |
| Individual 1 | 126.35567 | 0.01977 | 35.06046 | 96.12% |
| Individual 1 | 129.32046 | 0.01501 | 46.17970 | 91.74% |

The implementation of the nonlinear regression equation of the heart rate:

Various programming languages or statistical software can be used to calculate the nonlinear regression equation of the heart rate, such as C++, Java, Python, SAS, or even Excel. In all the embodiments of the present invention, the programming language Python is used to achieve the nonlinear regression equation the heart rate. However, it should be understood that the programming languages or statistical software are only the exemplary embodiments of the present invention, and should not be regarded as limiting.

Firstly, the programming language Python is used to find the “optimal natural logarithmic recessive equation”. Generally, the method of least squares is used. The method of least squares is a mathematical optimization technique which looks for the optimal matching function by minimizing the sum of the squares of the errors. Currently, there are a number of libraries of the method of least squares available on the market. Therefore, instead of creating a library by ourselves, an appropriate library can be selected from the market.

In an embodiment of the present invention, the curve_fit fucntion in SciPy library of Python is used. The curve_fit fucntion has a “fitting” feature. In addition, the callback function, in which the equation is the “optimal natural logarithmic recessive equation”, is also utilized. By applying the callback function to the curve_fit function, the curve_fit function repeatedly fits to or approaches the “optimal natural logarithmic recessive equation” of the callback function for obtaining the optimal equation curve of the heart rate based upon the data of time and the heart rate. The specific codes of the implementation are exampled as the following:

def fitFunc(t,a,b,c):

return a*np.exp(−b*t)+c

fitParams,fitCovariances=scipy.curve_fit(fitFunc,t,data) Formula 6:

The fitFunc is the callback function, and four parameters (arguments) are required to enter the function: which are time (t), the difference (a) between the heart rate at the initiation of the recovery period and the resting heart rate, the parameter (b) representing the heart rate recovery rate of the individual, and the resting heart rate (c). Three parameters (arguments) are passed to the curve_fit function: which are respectively the fitFunc, time (t), and heart rate date (HR). The other operational details and implementations of the recession equations can be easily deduced based upon the disclosure of the specification, and are not redundantly described herein. It should be appreciated that the implementations of the nonlinear regression equations of the heart rate described above are only the exemplary embodiments of the present invention, and should not be regarded as limiting.

The accuracy comparison between the linear regression equation and the nonlinear regression equation:

The accuracy of the heart rate recovery rate after the physical activity calculated by the linear regression equation and an nonlinear regression equation is compared herein. In the comparison of these embodiments, the nonlinear regression equation includes the exponential regression equation and the logarithmic regression equation. In the comparison of these embodiments, the coefficient of determination, also called the R-square, is used as the criteria for determination of accuracy.

In a regression mode constructed by the independent variable X and the dependent variable Y in a regression analysis, the coefficient of determination is used to understand to what extent the variable Y is decided and influenced by the variable X.

In the embodiments, since the independent variable X is time (t), and the dependent variable Y is heart rate (HR), the R-square represents how deterministic t is to HR herein. The maximum of R-square is 1, and the minimum thereof is 0. The higher the R-square is, the more decisive the independent variable of the present invention is to the dependent variable, the less influential the other unknown independent variables are to the dependent variable. The R-square can also be used to explain how much conformity of the calculated nonlinear regression equation is to the measured curve. Therefore, the higher R-square represents the greater significance of the parameter K of the present invention to the curve of the heart rate data.

R value is calculated according to the following formula:

$${SS}_{tot} = {\sum }_{i} {( {y}_{i} - \overline{y} )}^{2} , ( total sum of squares ) \\ {SS}_{res} = {\sum }_{i} {( {y}_{i} - {f}_{i} )}^{2} ( residual sum of squares ) {R}^{2} ≡ 1 - \frac{{SS}_{res}}{{SS}_{tot}} . \qquad Formula 7$$

Python can also be used to calculate R-square, and the specific codes of the implementation are exampled as the following:

for elem in data:

SSTo+=np.square(elem−AVG)

SSResid+=np.square(elem−fitFunc(t[index],fitParams[0],fitParams[1], fitParams[2]))

print “R-square”,1−SSResid/SSTo Formula 8:

The calculations of R-square described above are only the exemplary embodiments of the present invention, and should not be regarded as limiting.

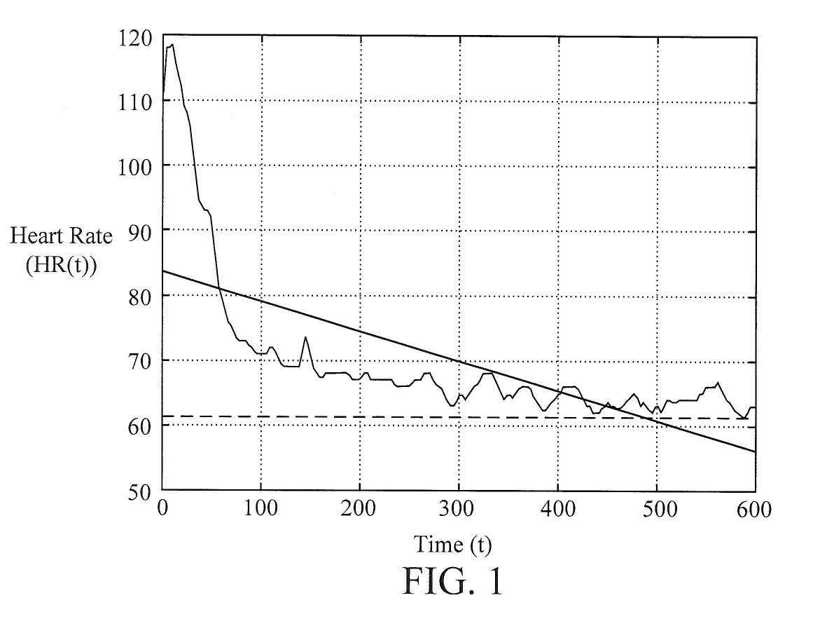

Please refer to FIG. 1 and FIG. 3, which are respectively a schematic graph showing the calculation of a heart rate recovery rate after a physical activity by using a linear repression equation in conventional techniques, and a schematic graph showing the calculation of a heart rate recovery rate after a physical activity by using an exponential regression equation in accordance with an embodiment of the invention. The horizontal axis represents time, the vertical axis represents the heart rate, the tortuous curve represents the heart rate data of an individual, the straight line represents the linear regression equation calculated based on the heart rate data, and the smooth curve represents the exponential regression equation calculated based on the heart rate data. In the figures, the R-square of the linear regression equation is 0.4458, and the R-square of the exponential regression equation is 0.9612. Please refer to Table 2, which shows the R-square averages of various regression equations. The R-square of the nonlinear regression equation is significantly higher than the linear regression equation, wherein the accuracy of the exponential regression equation (0.8852) is far higher than the linear regression equation (0.5743). The reason is as follows: in FIG. 3, the heart rate during the early phase of the recovery period rapidly declines, and the heart rate during the late phase of the recovery period gradually approaches the horizontal line of the resting hear rate. The exponential regression line closely conforms to the curve of the heart rate data. Conversely, in FIG. 1, the conventional linear regression equation only loosely conforms with the curve of the heart rate data during the late phase of the recovery period, while significantly deviates from the curve of the heart rate data during the early phase of the recovery period. The accuracy of the exponential regression equation is far higher than the linear regression equation.

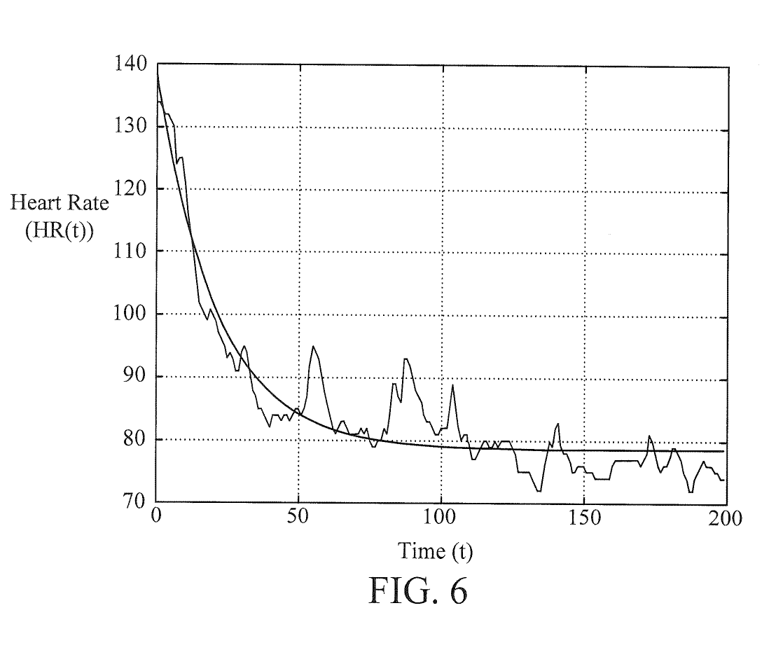

Please refer to FIG. 6 and FIG. 4, which are respectively a schematic graph showing the calculation of a heart rate recovery rate after a physical activity by using an exponential regression equation in accordance with another embodiment of the invention, and a schematic graph showing the calculation of a heart rate recovery rate after a physical activity by using an logarithmic regression equation in accordance with an embodiment of the invention. The horizontal axis represents time, the vertical axis represents the heart rate, the tortuous curve represents the heart rate data of an individual, and the smooth curve represents the nonlinear regression equation calculated based on the heart rate data. In the figures, the R-square of the exponential regression equation is 0.9005, and the R-square of the logarithmic regression equation is 0.8600. Please refer to Table 2, which shows the R-square averages of various regression equations. The R-square of the exponential regression equation (0.8852) is slightly higher than the logarithmic regression equation (0.8242). The reason is as follows: in FIG. 6, the heart rate during the early phase of the recovery period rapidly declines, and the heart rate during the late phase of the recovery period gradually approaches the horizontal line of the resting hear rate. The exponential regression line closely conforms to the curve of the heart rate data. Conversely, in FIG. 4, the logarithmic regression equation can closely conforms with the curve of the heart rate data during the early phase of the recovery period, but rather than gradually approaches the horizontal line of the resting hear rate, the logarithmic regression equation gradually declines and slightly deviates from the horizontal line of the resting hear rate during the late phase of the recovery duration. Although the accuracy of the logarithmic regression equation is slightly lower than the exponential regression equation, the logarithmic regression equation is still accurate.

TABLE 2

| | R-square |
|---|---|
| Regression equations | (coefficient of determination) |
| Exponential | 0.8852 |
| Logarithmic | 0.8242 |
| Linear | 0.5743 |

An apparatus of the present invention for measuring a physical condition:

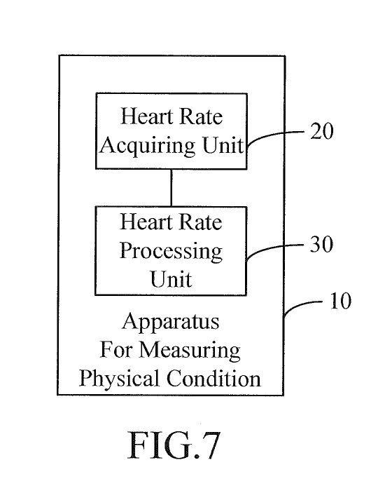

Please refer to FIG. 7, a structural schematic diagram of an apparatus for measuring a physical condition (e.g. the cardiopulmonary function/autonomic nervous regulation condition) of an individual by using a heart rate recovery rate after a physical activity in accordance with an embodiment of the invention is illustrated.

In an embodiment of the present invention, an apparatus 10 for measuring a physical condition is provided in the present invention, which includes a heart rate acquiring unit 20 and a heart rate processing unit 30. The heart rate acquiring unit is used for acquiring a heart rate of an individual during a recovery period after a physical activity conducted by the individual, and outputting digitized signals of the heart rate. For instance, the heart rate acquiring unit 20 can be a ECG signal detector, a heart sound detector, and a pulse detector.

When the heart rate acquiring unit 20 is an ECG signal detector, the heart rate acquiring unit 20 may include a or several pairs of electrodes to be placed on the skin of an individual for measuring the ECG signals of the individual. Increasing the number of electrodes can increase the accuracy of measurement, but this does not mean that only using a single pair of electrodes in the heart rate measurement unit is not feasible, nor should thus be regarded as limiting. When the heart rate acquiring unit 20 is a heart sound detector, the heart rate acquiring unit 20 may include a sound sensor for sensing heart sounds transmitted through the body of an individual. When the heart rate acquiring unit 20 is a pulse detector, the heart rate measurement unit 20 may include a vibration sensor for sensing the pulse of an individual. The heart rate processing unit 30 may be a processor connected to the heart rate acquiring unit 20 for receiving the digitized signals of the heart rate. The heart rate processing unit 30 has at least an arithmetic logic unit for calculating the nonlinear regression equation of the heart rate of the individual, which includes a parameter representing the heart rate recovery rate, based upon the heart rate of the individual, and determining the physical condition (e.g. the cardiopulmonary function/autonomic nervous regulation condition) of the individual based upon the parameter. Moreover, the apparatus 10 for measuring a physical condition can further include a memory unit and a display for recording and displaying the information, such as the measured heart rate data, the regression equations, the parameter K representing the heart rate recovery rate of the individual after the physical activity, the heart rate recovery half life t_{1/2}, the physical condition (e.g. the cardiopulmonary function/autonomic nervous regulation condition), and so on. Since the technical features of the present invention can be easily applied to various apparatuses based upon the disclosure of the specification of the present invention, the detailed embodiments are not redundantly described herein. The implementations of the elements described above are only used as the exemplary embodiments of the present invention, and should not be regarded as limiting.

In summary, the technical feature of the present invention is to calculate a heart rate recovery rate after a physical activity by using a nonlinear regression equation for resolving the inaccuracy problem resulted from the calculation of the heart rate recovery rate after the physical activity by using a linear regression equation in prior arts.

The terms “comprises”, “comprising”, “includes”, “including”, “having”, and their conjugates mean “including but not limited to”.

As used herein, the singular form “a”, “an”, and “the” include plural references unless the context clearly dictates otherwise.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range.

Various embodiments and aspects of the present invention as delineated hereinabove and as claimed in the claims section below find experimental support in the following examples.

The present invention has been described with a preferred embodiment thereof and it is understood that various modifications, without departing from the spirit of the present invention, are in accordance with the embodiments of the present invention. Hence, the embodiments described are intended to cover the modifications within the scope and the spirit of the present invention, rather than to limit the present invention.

It will be appreciated that the present invention is not limited to what has been particularly shown and described hereinabove. Rather the scope of the present invention includes both combinations and sub-combinations of various features described hereinabove as well as modifications thereof which would occur to a person of ordinary skill in the art upon reading the foregoing description and which are not in the prior art.

What is claimed is:

1. A method for measuring a physical condition by using a heart rate recovery rate, comprising steps of:
acquiring a heart rate of an individual during a recovery period after a physical activity conducted by the individual;
calculating a nonlinear regression equation of the heart rate of the individual, which includes a parameter representing the heart rate recovery rate of the individual, based upon the heart rate of the individual; and
determining the physical condition of the individual based upon the parameter.

2. The method as claimed in claim 1, wherein the nonlinear regression equation comprises an exponential regression equation or a logarithmic equation.

3. The method as claimed in claim 2, wherein the exponential regression equation is defined as the following equation:
HR(t)=HR_{d}×C^{(-K×t)}+HRᵣ; or
HR=(HR₀−HRᵣ)×C^{(-K×t)}+HRᵣ,
wherein t is time, HR(t) is the heart rate at the time t, HRᵣ is a resting heart rate, HR₀ is a heart rate at the initiation of the recovery period, HR_{d} is a difference between the heart rate at the initiation of the recovery period and the resting heart rate, K is a parameter representing the heart rate recovery rate of the individual, and C is a constant.

4. The method as claimed in claim 3, wherein the constant C is a natural logarithm e.

5. The method as claimed in claim 3, wherein a relationship between a heart rate recovery half life and the parameter K representing the heart rate recovery rate of the individual is defined as the following equation:
$${t}_{1 / 2} = \frac{{log}_{c} 2}{K} ,$$
wherein t_{1/2} is the heart rate recovery half life.

6. The method as claimed in claim 2, wherein the logarithmic regression equation is defined as the following equation:
HR(t)=a×log_{c}t+R,
wherein t is time, HR(t) is the heart rate at the time t, R is a parameter associated with a resting heart rate, a is a parameter representing the heart rate recovery rate of the individual, and C is a constant.

7. The method as claimed in claim 6, wherein the constant C is a natural logarithm e.

8. The method as claimed in claim 1, wherein the physical activity conducted by the individual is step aerobics.

9. The method as claimed in claim 1, wherein the step of acquiring a heart rate of the individual during a recovery period after a physical activity conducted by the individual comprises:
continuously acquiring the heart rate of the individual during the recovery period after the physical activity conducted by the individual.

10. The method as claimed in claim 1, wherein the physical condition is a cardiopulmonary function condition or an autonomic nervous control condition.

11. An apparatus for measuring a physical condition by using a heart rate recovery rate, comprising:
a heart rate acquiring unit configured to acquire a heart rate of an individual during a recovery period after a physical activity conducted by the individual, and to output a digitized signal of the heart rate; and
a heart rate processing unit connected with the heart rate acquiring unit to receive the digitized signal of the heart rate, to calculate a nonlinear regression equation of the heart rate of the individual, which includes a parameter representing the heart rate recovery rate, based upon the heart rate of the individual, and to determine the physical condition of the individual based upon the parameter.

12. The apparatus as claimed in claim 11, wherein the nonlinear regression equation comprises an exponential regression equation or a logarithmic equation.

13. The apparatus as claimed in claim 12, wherein the exponential regression equation is defined as the following equation:
HR(t)=HR_{d}×C^{(-K×t)}+HRᵣ; or
HR=(HR₀−HRᵣ)×C^{(-K×t)}+HRᵣ,
wherein t is time, HR(t) is the heart rate at the time t, HRᵣ is a resting heart rate, HR₀ is a heart rate at the initiation of the recovery period, HR_{d} is a difference between the heart rate at the initiation of the recovery period and the resting heart rate, K is a parameter representing the heart rate recovery rate of the individual, and C is a constant.

14. The apparatus as claimed in claim 13, wherein the constant C is a natural logarithm e.

15. The apparatus as claimed in claim 13, wherein a relationship between a heart rate recovery half life and the parameter K representing the heart rate recovery rate of the individual is defined as the following equation:
$${t}_{1 / 2} = \frac{{log}_{c} 2}{K} ,$$
wherein t_{1/2} is the heart rate recovery half life.

16. The apparatus as claimed in claim 14, wherein the logarithmic regression equation is defined as the following equation:
HR(t)=a×log_{c}t+R,
wherein t is time, HR(t) is the heart rate at the time t, R is a parameter associated with a resting heart rate, a is a parameter representing the heart rate recovery rate of the individual, and C is a constant.

17. The apparatus as claimed in claim 11, wherein the physical activity conducted by the individual is step aerobics.

18. The apparatus as claimed in claim 11, wherein the heart rate acquiring unit continuously acquires the heart rate of the individual during the recovery period after the physical activity conducted by the individual.

19. The apparatus as claimed in claim 11, wherein the apparatus is a wearable apparatus.

20. The apparatus as claimed in claim 11, wherein the physical condition is a cardiopulmonary function condition or an autonomic nervous control condition.
